(19) United States
(12) Patent Application Publication
(10) Pub. No.: US 2018/0207417 A1
(43) Pub. Date: Jul. 26, 2018

(54) SELF-ALIGNMENT MECHANISM FOR IMAGING CATHETER AND DRIVE ASSEMBLY

(71) Applicant: AVINGER, INC., Redwood City, CA (US)

(72) Inventors: Michael ZUNG, San Carlos, CA (US); Douglas Joseph Scott BOURNE, Campbell, CA (US)

(21) Appl. No.: 15/741,773

(22) PCT Filed: Jul. 6, 2016
(86) PCT No.: PCT/US2016/041193

§ 371 Date: Jan. 4, 2018

Related U.S. Application Data
(60) Provisional application No. 62/189,077, filed on Jul. 6, 2015.

(51) Int. Cl. A61M 39/10; A61B 5/00

(52) U.S. Cl. CPC A61M 39/10; A61B 5/0066; A61B 5/02007; A61B 2562/228; A61B 5/6852; A61B 5/0084

(57) ABSTRACT

A self-aligning system for coupling a catheter to a rotational drive assembly includes a catheter and a drive assembly. The catheter includes an elongate catheter body, a rotatable driveshaft extending within the elongate catheter body, a connector at a proximal end of the elongate catheter body, and a keyed feature extending from an outer diameter of the connector. The drive assembly includes a motor configured to rotate the driveshaft, a receiving tube with a distal opening configured to receive the connector, and a channel on an inner surface of the receiving tube, the channel including two opposing curved walls that are angled towards a substantially straight section. The keyed feature is configured to slide against one of the opposing curved walls and into the substantially straight section to rotationally align the proximal end of the catheter with the drive assembly.

### CROSS REFERENCE TO RELATED APPLICATIONS

This application claims priority to U.S. Provisional Patent Application No. 62/189,077, filed Jul. 6, 2015, and titled “CATHETER/SLED SELF-ALIGNMENT FEATURE,” which is incorporated by reference in its entirety.

This application may also be related to U.S. patent application Ser. No. 14/400,151, filed Nov. 10, 2014, titled “ATHERECTOMY CATHETER DRIVE ASSEMBLIES,” now U.S. Pat. No. 9,345,398, which is incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

Described herein are self-aligning systems for connecting an optical fiber of an imaging catheter, such as an optical coherence tomography (OCT) imaging catheter, with an optical fiber of a drive assembly configured to rotate the optical fiber of the imaging catheter.

### BACKGROUND

Optical coherence tomography (OCT) catheters, such as atherectomy catheters, have been developed for use in treatment of the coronary vasculature, including the peripheral vasculature. On-board OCT may be used to visualize and guide treatment with the catheter, such as for removal of plaque during an atherectomy procedure.

During operation of an OCT catheter, a light source outside the catheter may be used to introduce light into the delivery fiber. A detector, also outside the catheter, which may include an interferometer, detects light from the fiber and generates an electrical signal representative of that light. This signal is then digitized and provided for analysis. It may be particularly desirable to circumferentially scan the light around the vessel wall, which may be done by rotating the fiber and (in some variations) at least a portion of the catheter about its axis. However, since neither the light source nor the processor spin with the catheter, it may be difficult to couple light into and out of the optical fibers and continuously drive the rotation of the catheter using a drive assembly.

The fiber of an OCT catheter needs be coupled with the drive assembly in a specifically oriented manner so that signals can be processed correctly. In addition, the fiber and/or rotational drive shaft must be driven with sufficient power to reliably rotate the device. Currently available drive assembly and catheter coupling engagements require the optical connector of the catheter to be positioned in a specific orientation, e.g., a top-dead-center orientation, when attached to the drive assembly. In turn, the drive assembly optical connection also needs to be positioned in a mating orientation, e.g., a top-dead-center orientation.

Catheter connector orientation, however, may be dependent on the manufacturing process to properly position it when assembled. The drive assembly connector orientation may also be dependent on the internal flag and sensor components positioning the connector in the proper and compatible orientation. This co-alignment of both independent devices can at times be inconsistent and inconvenient for the user, resulting in failed connection. As a result, the optical connector orientation within the drive assembly of current systems often requires manual correction during use.

Described herein are coupling connections between a catheter (e.g., an OCT catheter) and a drive assembly and methods of operating them that may address some or all of these issues.

### SUMMARY OF THE DISCLOSURE

In general, in one embodiment, a self-aligning system for coupling a catheter to a rotational drive assembly includes a catheter and a drive assembly. The catheter includes an elongate catheter body, a rotatable driveshaft extending within the elongate catheter body, a connector at a proximal end of the elongate catheter body, and a keyed feature extending from an outer diameter of the connector. The drive assembly includes a motor configured to rotate the driveshaft, a receiving tube with a distal opening configured to receive the connector, and a channel on an inner surface of the receiving tube, the channel including two opposing curved walls that are angled towards a substantially straight section. The keyed feature is configured to slide against one of the opposing curved walls and into the substantially straight section to rotationally align the proximal end of the elongate catheter body with the drive assembly.

In general, in one embodiment, a catheter includes an elongate catheter body, a rotatable driveshaft extending within the elongate catheter body, a connector at a proximal end of the elongate catheter body, and a keyed feature extending from an outer diameter of the connector. The catheter is configured to connect to a drive assembly for rotating the rotatable drive shaft. The keyed feature is configured to slide against opposing curved walls of the drive assembly and into a substantially straight section to rotationally align a proximal end of the elongate catheter body with the drive assembly.

In general, in one embodiment, a drive assembly includes a motor configured to rotate a catheter driveshaft, a receiving tube with a distal opening configured to receive a connector of a catheter, and a channel on an inner surface of the receiving tube. The channel includes two opposing curved walls that are angled towards a substantially straight section. The opposing curved walls are configured to receive a keyed feature of the catheter and guide the keyed feature into the substantially straight section to rotationally align the proximal end of the catheter with the drive assembly.

This and other embodiments can include one or more of the following features. The self-aligning system can further include a first optical fiber extending through the elongate catheter body and a second optical fiber within the driveshaft configured to transfer light from a light source to the optical fiber of the catheter when optically connected thereto. Rotationally aligning the proximal end of the catheter with the drive assembly can optically couple the first optical fiber with the second optical fiber. The drive assembly can further include a fiber optic rotating junction having a stationary portion with a stationary fiber therein and a rotatable portion with a rotatable fiber therein. The motor can be configured to rotate the rotatable portion of the fiber optic rotating junction. The motor can be hollow and configured to house a portion of the fiber optic rotating junction of the drive assembly such that the motor and the fiber optic rotating junction are coaxial. A distal end of the receiving tube can be beveled. The bevel can be at an angle of between 35 and 45 degrees relative to a central longitudinal axis of the receiving tube. The bevel can include a tip. The straight section of the channel can be approximately 180 degrees from the tip. The keyed feature can be a cylindrical post extending from the connector. A central long axis of the cylindrical post can be substantially perpendicular to a central long axis of the connector. The connector can be substantially cylindrical. An inner diameter of the receiving tube can be larger than an outer diameter of the connector without the keyed feature and smaller than an outer diameter of the connector with the keyed feature. An inner surface of the receiving tube can further include a first notched tab and a second notched tab positioned on opposite sides of the substantially straight section.

In general, in one embodiment, a self-aligning coupling apparatus for coupling an imaging device to a rotational drive assembly includes an imaging device and a drive assembly. The imaging device includes an elongate body, a first optical fiber extending through the catheter body, a connector at a proximal end of the elongate body, and a keyed feature extending from an outer diameter of the connector. The drive assembly includes a motor, a second optical fiber, a receiving tube with a distal opening, and a channel in an inner surface of the receiving tube. The motor is configured to rotate the optical fiber. The second optical fiber is configured to transfer light from a light source to the optical fiber of the imaging device when optically connected thereto. The receiving tube with a distal opening is configured to receive the connector. The channel includes two opposing curved walls that are angled towards a substantially straight section. The keyed feature is configured to slide against one of the opposing curved walls and into the substantially straight section to align the proximal end of the imaging device with the drive assembly to optically couple the first optical fiber with the second optical fiber.

In general, in one embodiment, an imaging device includes an elongate body, an optical fiber extending within the elongate body, a connector at a proximal end of the elongate body, and a keyed feature extending from an outer diameter of the connector. The imaging device is configured to connect to a drive assembly for rotating the optical fiber. The keyed feature is configured to slide against opposing curved walls of the drive assembly and into a substantially straight section to rotationally align the proximal end of the elongate body with the drive assembly to optically couple the optical fiber with a second optical fiber of the drive assembly.

In general, in one embodiment, a drive assembly includes a motor, a receiving tube with a distal opening, an optical fiber, and a channel on an inner surface of the receiving tube. The motor is configured to rotate an imaging device. The receiving tube with a distal opening is configured to receive a connector of the imaging device. The optical fiber is configured to transfer light from a light source to an optical fiber of the imaging device when optically connected thereto. The channel includes two opposing curved walls that are angled towards a substantially straight section. The opposing curved walls are configured to receive a keyed feature of the imaging device and guide the keyed feature into the substantially straight section to rotationally align the proximal end of the imaging device with the drive assembly to optically couple the optical fiber of the drive assembly with the optical fiber of the imaging device.

In general, in one embodiment, a self-aligning system for coupling a catheter to a rotational drive assembly includes a catheter and a drive assembly. The catheter includes an elongate catheter body a rotatable driveshaft extending within the elongate catheter body, a connector at a proximal end of the elongate catheter body, and a keyed feature extending from an outer diameter of the connector. The drive assembly includes a motor configured to rotate the driveshaft, a receiving tube with a distal opening configured to receive the connector, and a channel on an inner surface of the receiving tube. The channel includes a curved wall that spirals to a substantially straight section. The keyed feature is configured to slide along the curved wall and into the substantially straight section to rotationally align the proximal end of the elongate catheter body with the drive assembly.

In general, in one embodiment, a catheter includes an elongate catheter body, a rotatable driveshaft extending within the elongate catheter body, a connector at a proximal end of the elongate catheter body, and a keyed feature extending from an outer diameter of the connector. The catheter is configured to connect to a drive assembly for rotating the rotatable drive shaft. The keyed feature is configured to slide against a curved wall of the drive assembly and into a substantially straight section to rotationally align the proximal end of the elongate catheter body with the drive assembly.

In general, in one embodiment, a drive assembly includes a motor, a receiving tube with a distal opening, and a channel on an inner surface of the receiving tube. The motor is configured to rotate a catheter driveshaft. The receiving tube with a distal opening is configured to receive a connector of a catheter. A channel on an inner surface of the receiving tube includes a curved wall that is angled towards a substantially straight section. The curved wall is configured to receive a keyed feature of the catheter and guide the keyed feature into the substantially straight section to rotationally align the proximal end of the catheter with the drive assembly.

In general, a self-aligning coupling apparatus for coupling an imaging device to a rotational drive assembly includes an imaging device and a drive assembly. The imaging device includes an elongate body, a first optical fiber extending through the catheter body, a connector at a proximal end of the elongate body, and a keyed feature extending from an outer diameter of the connector. The drive assembly includes a motor, a second optical fiber, a receiving tube with a distal opening, and a channel on an inner surface of the receiving tube. The motor is configured to rotate the optical fiber. The second optical fiber is configured to transfer light from a light source to the optical fiber of the imaging device when optically connected thereto. The receiving tube with a distal opening is configured to receive the connector. The channel on an inner surface of the receiving tube includes a curved wall that is angled towards a substantially straight section. The keyed feature is configured to slide against the curved wall and into the substantially straight section to align the proximal end of the imaging device with the drive assembly to optically couple the first optical fiber with the second optical fiber.

In general, in one embodiment, an imaging device includes an elongate body, an optical fiber extending within the elongate body, a connector at a proximal end of the elongate catheter body, and a keyed feature extending from an outer diameter of the connector. The imaging device is configured to connect to a drive assembly for rotating the optical fiber. The keyed feature is configured to slide against a curved wall of the drive assembly and into a substantially straight section to rotationally align the proximal end of the elongate body with the drive assembly to optically couple the optical fiber with a second optical fiber of the drive assembly.

In general, in one embodiment, a drive assembly includes a motor, a receiving tube with a distal opening, an optical fiber and a channel on an inner surface of the receiving tube. The motor is configured to rotate an imaging device. The receiving tube with a distal opening is configured to receive a connector of an imaging device. The optical fiber is configured to transfer light from a light source to an optical fiber of the imaging device when optically connected thereto. The channel on an inner surface of the receiving tube includes a curved wall that is angled towards a substantially straight section. The curved wall is configured to receive a keyed feature of the imaging device and guide the keyed feature into the substantially straight section to rotationally align the proximal end of the catheter with the drive assembly to optically couple the optical fiber of the drive assembly with the optical fiber of the imaging device.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 is an example of a proximal end of an imaging catheter having an elongate body connector and a tapered elongate keyed feature extending therefrom.

FIG. 2 is an example of a drive assembly receiving tube having a receiving channel with opposed curved walls to drive the tapered end of the elongate keyed surface into alignment.

FIG. 3A is an example of a catheter handle having the proximal end with the keyed elongate body connector engaging with a drive assembly having a receiving tube as described herein.

FIG. 3B is another example showing an enlarged view from FIG. 3A of the proximal end of the handle cylinder engaging with the drive assembly tube, showing the start of the engagement elongate keyed surface engaging the curved inner channel wall of the drive assembly tube to guide the elongate keyed surface into the receiving tube.

FIG. 3C is shows the example of FIG. 3A with the catheter cylinder pushed proximally into the receiving tube of the drive assembly so that the elongate keyed feature is engaged and aligned relative to the drive assembly.

FIG. 4 is an example of a proximal end of an imaging catheter including a cylindrical keyed feature.

FIG. 5 is an example of a drive assembly receiving tube having a beveled distal end with curved walls and an elongate straight channel.

FIGS. 6A-6C show placement of the proximal end of the catheter of FIG. 4 within the receiving tube of FIG. 5. FIG. 6D shows a cross-section of the catheter of FIG. 4 within the receiving tube of FIG. 5.

FIGS. 7A-7B show the interior of an exemplary drive assembly as a catheter is connected thereto. FIG. 7A shows the catheter not yet connected to the drive assembly. FIG. 7B shows the catheter engaged with the drive assembly.

FIG. 8 shows an exemplary fiber management unit within a drive assembly.

FIGS. 9A-9C show an exemplary drive assembly including a FORJ and mating catheter. FIG. 9A shows the lid removed. FIG. 9B shows the outer housing removed. FIG. 9C shows inner portions of the drive assembly.

FIG. 10 shows another embodiment of a receiving tube of a drive assembly.

### DETAILED DESCRIPTION

Self-aligning connections between a catheter having on-board imaging, such as optical coherence tomography (OCT), and a drive assembly are described herein.

For example, any of the systems described herein may include a catheter having a proximal end including an elongate body connector and a keyed feature extending therefrom. The system may also include a receiving tube on the drive assembly (e.g., the distal end region of the drive assembly) that includes a distal opening to receive the proximal end of the catheter.

The receiving tube of the drive assembly can have an inner diameter that is just slightly larger than the outer diameter of the body of the proximal end of the catheter. Moreover, the receiving tube may also include a channel cut into the wall of the receiving tube. This receiving channel in the wall of the receiving tube is specifically configured to reliably and securely guide the engagement of the proximal end of the catheter when the proximal end of the catheter is manually inserted into the receiving tube of the drive assembly in any orientation.

Any of the catheters described herein can include an elongate body and a driveshaft extending therein for rotating an imaging sensor. The drive assemblies can, in turn, be configured to rotate the optical fiber and/or the driveshaft. Further, the drive assemblies can include optical components, such as an optical fiber or FORJ, configured to optically connect with the optical fiber of the catheter.

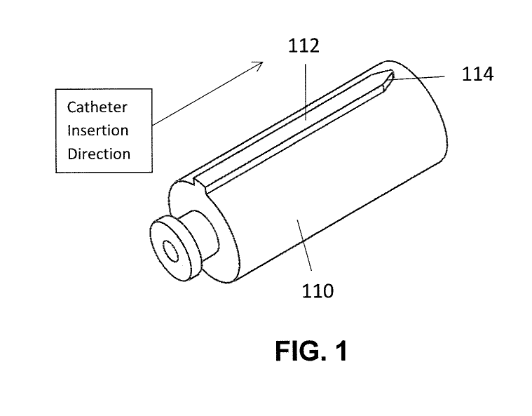

An exemplary self-alignment mechanism for a catheter and drive assembly is shown in FIGS. 1-3C. Referring to FIG. 1, the catheter can include a proximal end having an elongate body connector 110, such as a cylindrical elongate body. A keyed feature 112 can extend from the elongate body 110 (e.g., be proud of the elongate body). The keyed feature 112 extends along the length of the elongate body 110 and includes a tapered proximal end 114. Further, an optical fiber can extend through the elongate body connector 110 for optical connection with an optical component of the drive assembly.

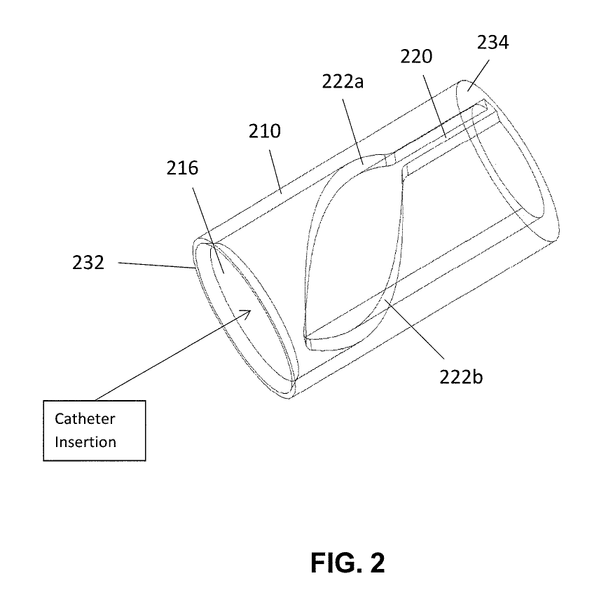

As shown in FIG. 2, the drive assembly can include a receiving tube 210. The smallest diameter of the tube 210 can be larger than the diameter of the elongate body 110 of the catheter. However, the receiving tube 210 can have a channel 216 cut into the inner surface thereof from the proximal end to the distal end to allow for travel of the keyed feature 112 therethrough. The channel 216 can have approximately the same depth (or larger) as the height of the keyed feature 112. Further, the channel 216 can have a diameter (around the inner circumference of the tube 210) that is greatest at the distal end 232 of the tube 210, but narrows as the channel 216 extends towards the proximal end 234. Thus, the channel 216 can include two opposed curved (e.g., helical) walls 222a,b that taper or come together at a narrow substantially straight or linear keyed section 220. The keyed section 220 can have a width that is approximately equal to the width of the keyed feature 112.

The keyed feature 112 on the proximal end of the catheter and the keyed section 220 can be positioned such that axial alignment of the two (i.e., placement of the keyed feature 112 within the keyed section 220 of the channel) will align the optical fiber of the catheter with the optical elements in the drive assembly.

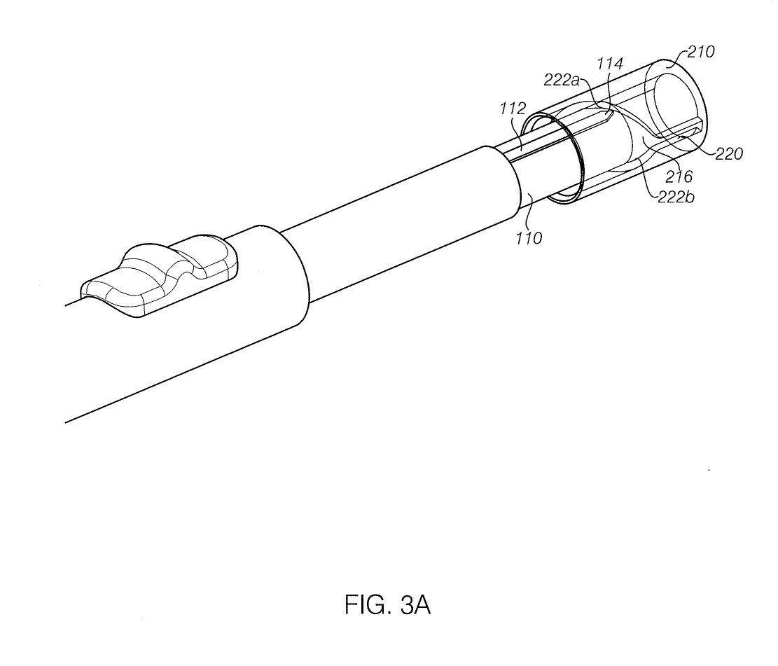

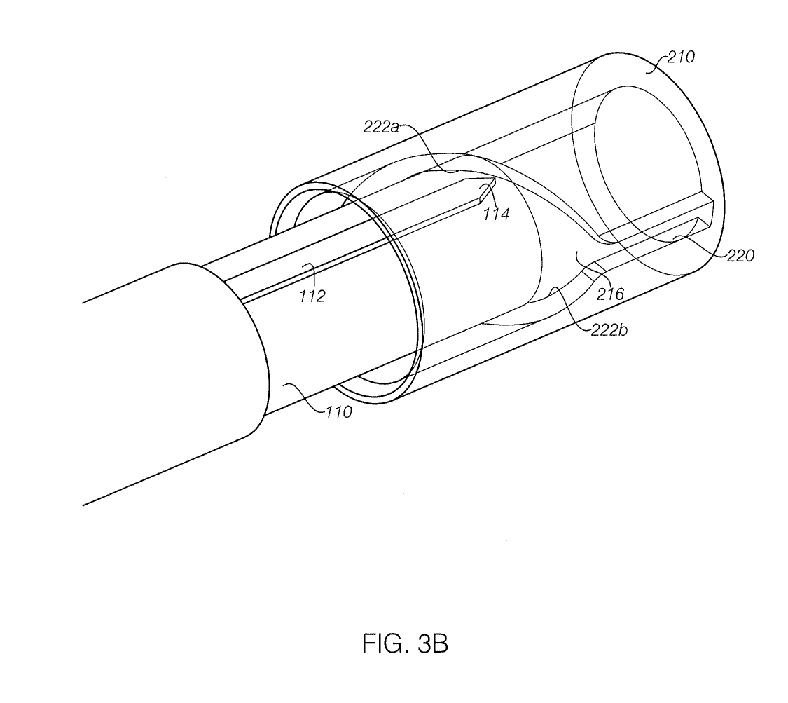

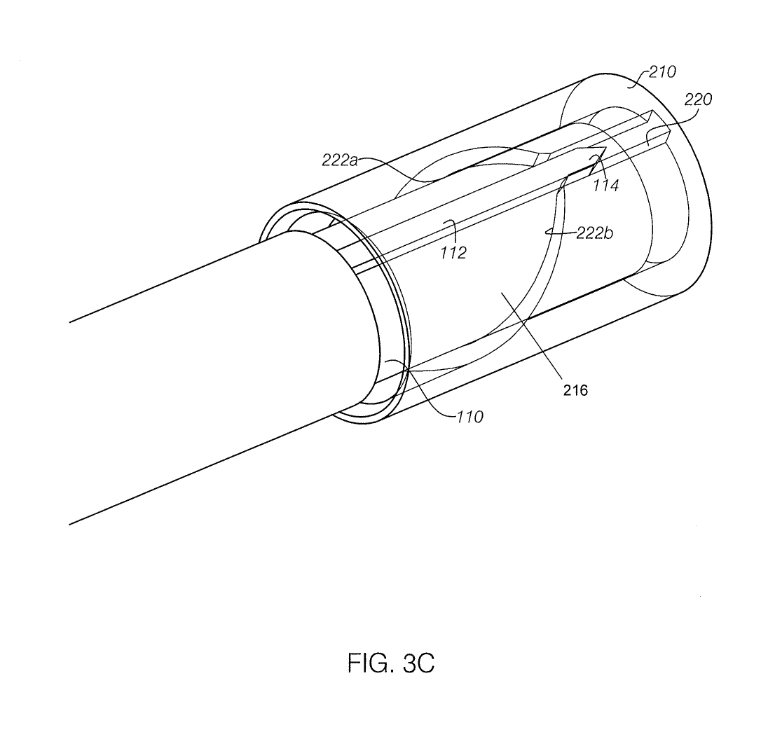

Thus, in use, referring to FIGS. 3A-3C, the elongate body connector 110 of the proximal end of the catheter can be inserted into the receiving tube 210 of the drive assembly with the keyed feature 112 at any radial position (e.g., 360° around) relative to the tube 210. The keyed feature 112 can be initially positioned in the wide mouth of the channel 216. As the elongate body connector 110 is pushed proximally into the tube 210, the tapered end 114 will contact one of the walls 222a,b of the channel 216. Continued pushing proximally will result in the tapered end 114 moving further proximally along the wall 222a,b. Because the walls 222a,b are curved towards the linear section 220, the keyed feature 112 and elongate body connector 110 will rotate relative to the receiving tube 210 (and/or the receiving tube 210 will rotate relative to the body 110) until the keyed feature 112 slides into the keyed section 220.

As a result, the proximal end of the catheter will be appropriately aligned with the drive assembly, therefore ensuring therefore ensuring proper optical alignment.

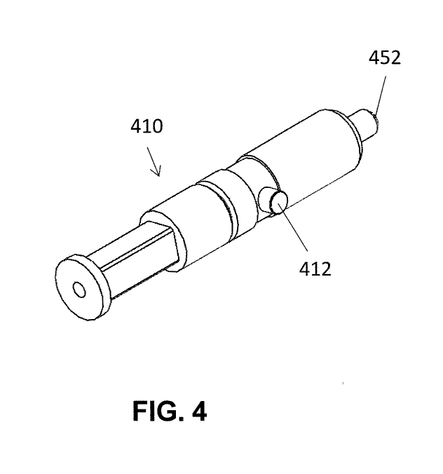

Another exemplary self-alignment mechanism for a catheter and drive assembly is shown in FIGS. 4-7B. Referring to FIG. 4, the catheter can include a proximal end having an elongate body connector 410, such as a substantially cylindrical elongate body. The optical fiber of the catheter can extend through the elongate body and terminate at the proximal tip 452. The proximal tip 452 can have a smaller diameter than the rest of the elongate body 410. For example, the proximal tip 452 can be, for example, an FC optical connector. In some embodiments, the tip 452 can further include a spring therein that is configured to absorb tolerance when mating the optical fiber of the catheter with the optical fiber of the drive assembly. The driveshaft of the catheter can likewise extend through the connector 410 to the proximal tip 452.

Further, a keyed feature 412 can extend from the elongate body connector 410 (e.g., be proud of the elongate body). The keyed feature 412 in this embodiment is a cylindrical post extending from the elongate body 410. The central long axis of the cylindrical post can be substantially perpendicular to the central long axis of the elongate body 410.

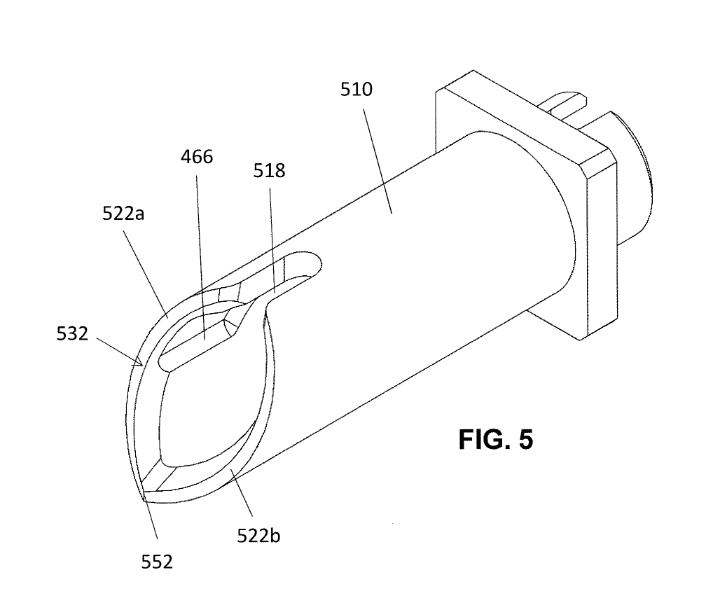

As shown in FIG. 5, the drive assembly can include a receiving tube 510. The inner diameter of the tube 510 can be larger than the outer diameter of the elongate body 410 of the catheter. The distal end 532 of the tube 510 can be beveled across the entire diameter thereof to form a tip 552. The angle of the bevel can be, for example, between 35 and 45 degrees, such as about 40 degrees, relative to the longitudinal axis of the receiving tube 510. Further, each of the opposing beveled walls 522a,b can be scooped or curved inwards (i.e., concave and/or helical). Further, the two walls 522a,b can come together at a narrow or straight keyed channel 520. The keyed channel 520 can be positioned at the low point of the bevel, i.e., be approximately 180° away from the tip 552. The keyed channel 520 can have a width that is approximately equal to the width or diameter of the keyed feature 412. In some embodiments, the tube 510 can included a continuous tapered or beveled wall 524 on the innermost diameter thereof to act as a funnel or direction feature to help guide the proximal end of the catheter therein.

The keyed feature 412 on the proximal end of the catheter and the keyed channel 520 can be positioned such that axial alignment of the two (i.e., placement of the keyed feature 412 within the linear channel 520) will align the optical fiber of the catheter with the optical elements in the drive assembly.

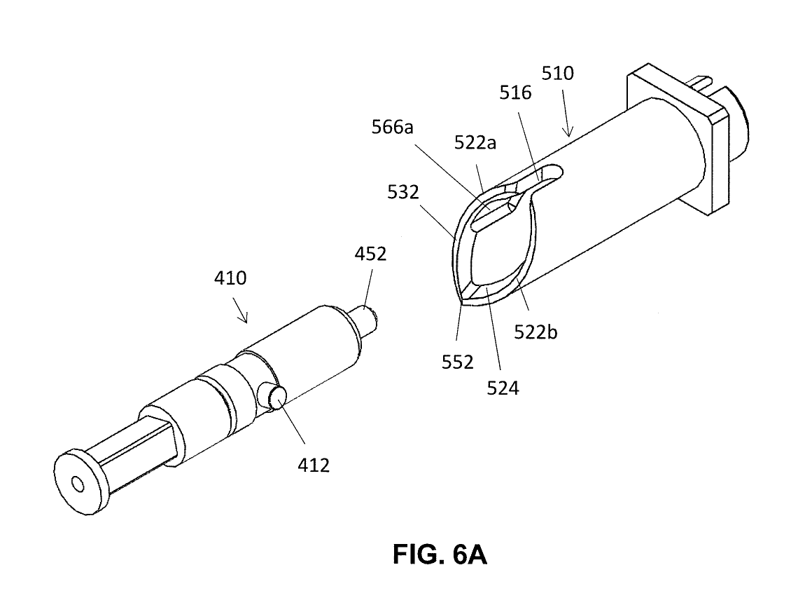

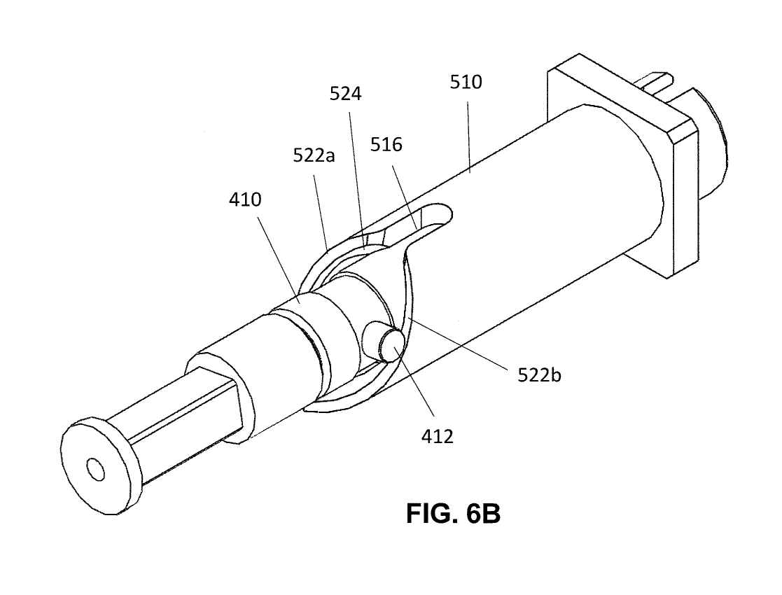

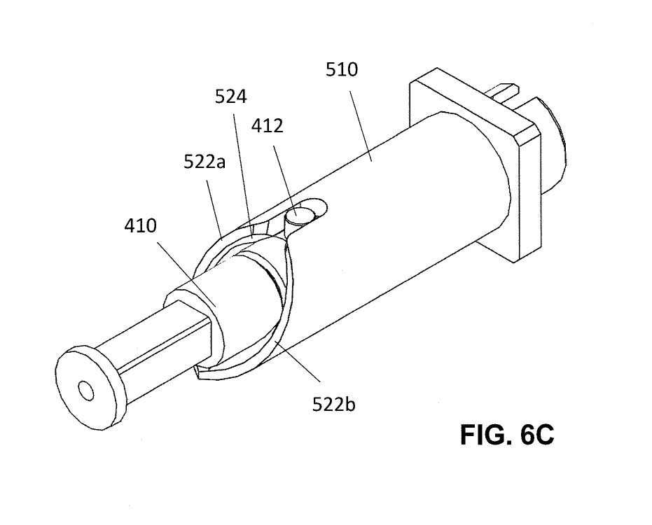

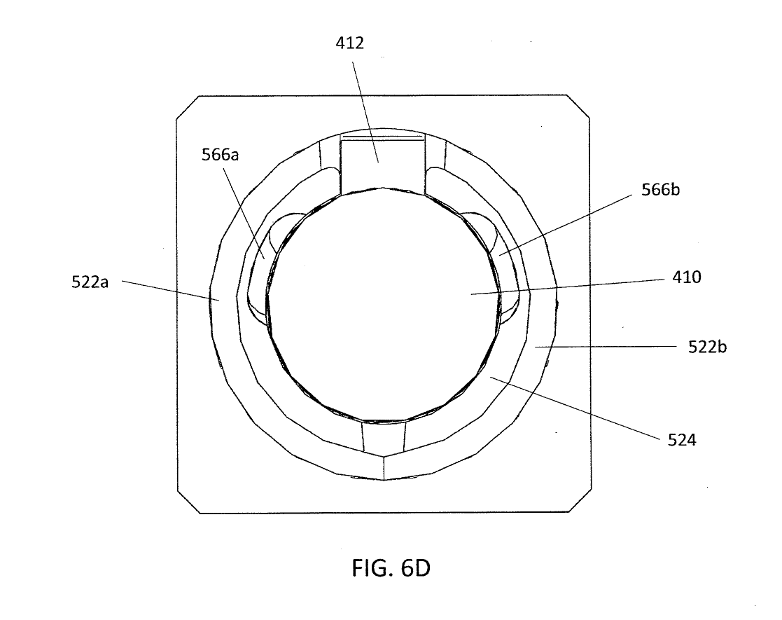

Thus, in use, referring to FIGS. 6A-6D, the elongate body 410 of the proximal end of the catheter can be inserted into the receiving tube 510 of the drive assembly with the keyed feature 412 at any radial position (e.g., 360° around) relative to the tube 510. As the elongate body 410 is pushed proximally into the tube 510, the keyed feature 412 will contact one of the walls 522a,b. Continued pushing proximally will result in feature 412 moving further proximally along the wall 522a,b. Because the walls 522a,b are curved towards the linear section 220, the keyed feature 412 and body 410 will rotate (and/or the receiving tube 510 will rotate relative to the body 410) until the keyed feature 412 slides into the keyed channel 520. As a result, the proximal end of the catheter will be appropriately aligned with the drive assembly, therefore ensuring proper alignment (e.g., for the desired optical connection thereto).

In some embodiments, the curved walls 522a,b have a curvature such that as the cylindrical feature 412 touches and slides along the walls 522a,b, it contacts or touches the walls 522a,b as a line.

In some embodiments, the inner surface of the receiving tube 510 can include one or more notched tabs 566a,b. For example, a notched tab 566a,b can extend on each side of the keyed channel 520, e.g., along the beveled wall 524. Each notched tab 566a,b can extend, for example, between 30 degrees and 60 degrees, e.g., 40 degrees-50 degrees around the circumference of the receiving tube. The tab 466 can be configured to prevent the keyed feature 412 from binding along the inner diameter of the receiving tube 510 as it rotates relative to the receiving tube 510.

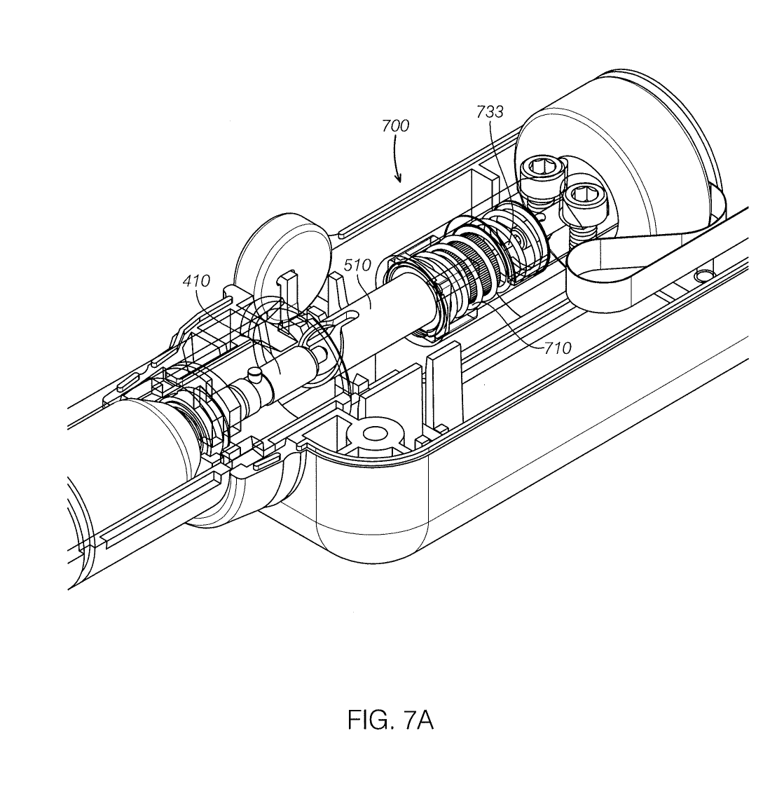

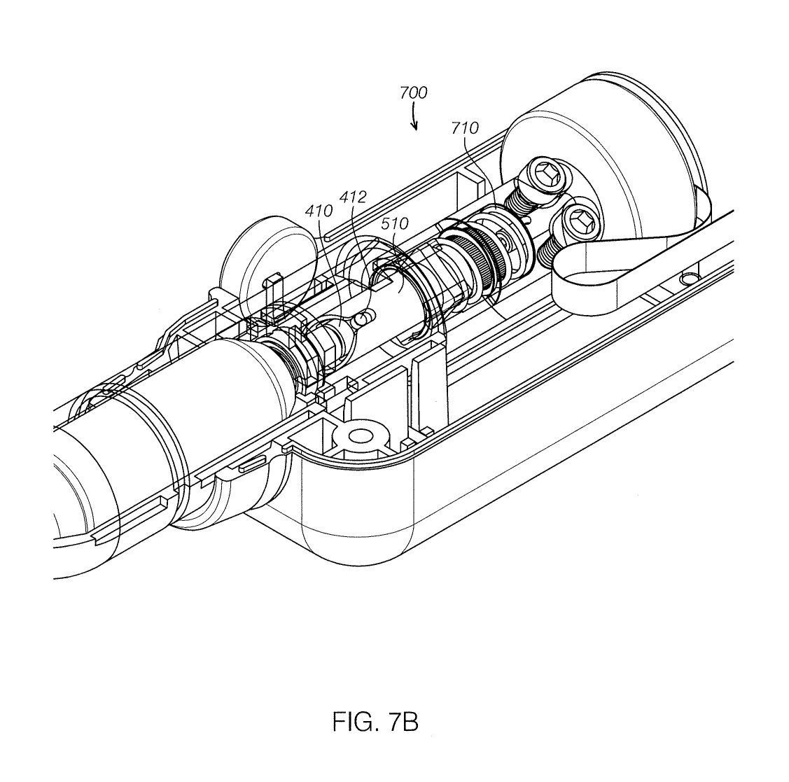

In some embodiments, referring to FIGS. 7A-7B, the drive assembly 700 can include a spring mechanism 770 therein. The spring mechanism 710 can be attached to the distal end 534 of the receiving tube 510. As the elongate body 410 is pushed into the receiving tube 510, the spring mechanism 710 can be configured to compress and hold the optical fibers against one another.

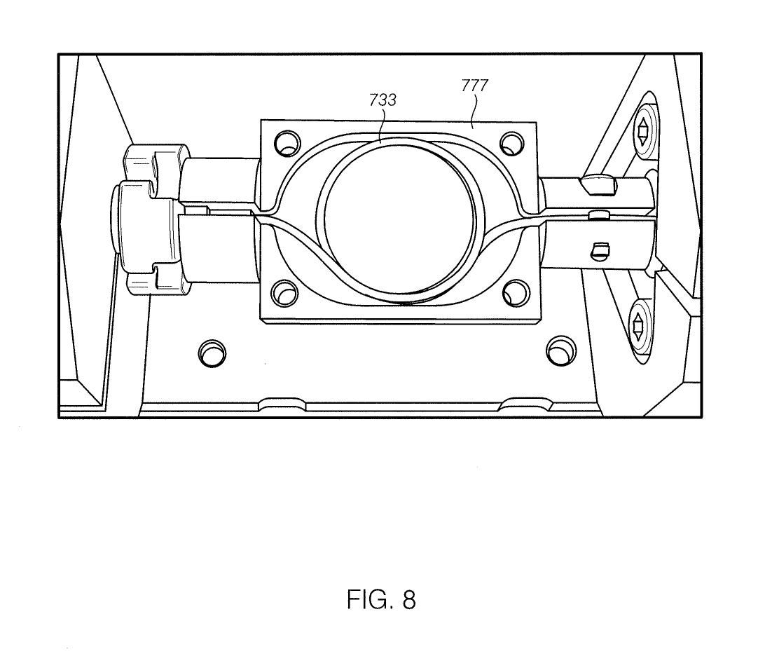

In some embodiments, when the spring mechanism 710 compresses, the optical fiber 733 in the drive assembly also moves proximally. In order to accommodate for the movement of the optical fiber, the drive assembly 700 can include a fiber management unit 777 (see, e.g., FIG. 8). The fiber management unit 777 can include a racetrack 776 or carved loop therein configured to allow the loop of fiber 773 to expand and contract (thereby releasing greater lengths of fiber 733 and/or reeling the fiber 733 in).

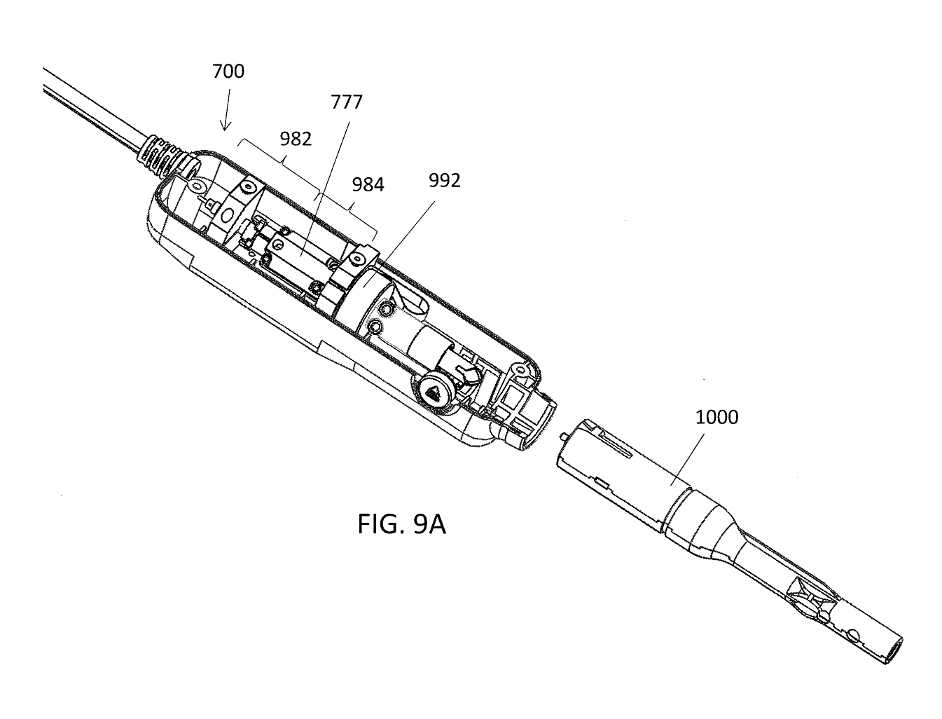

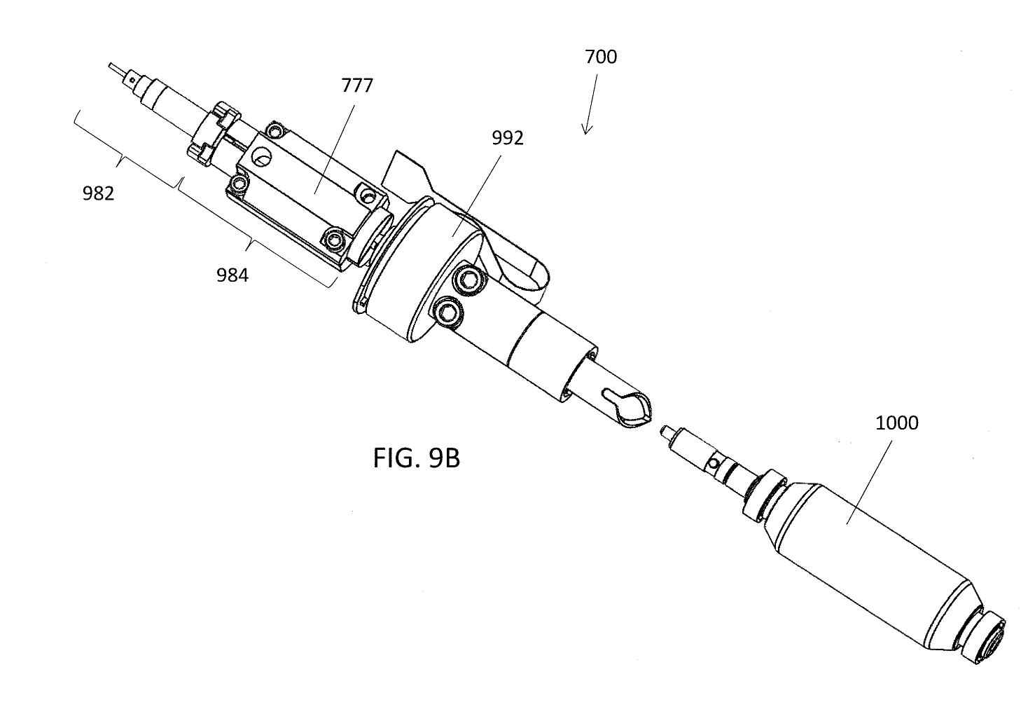

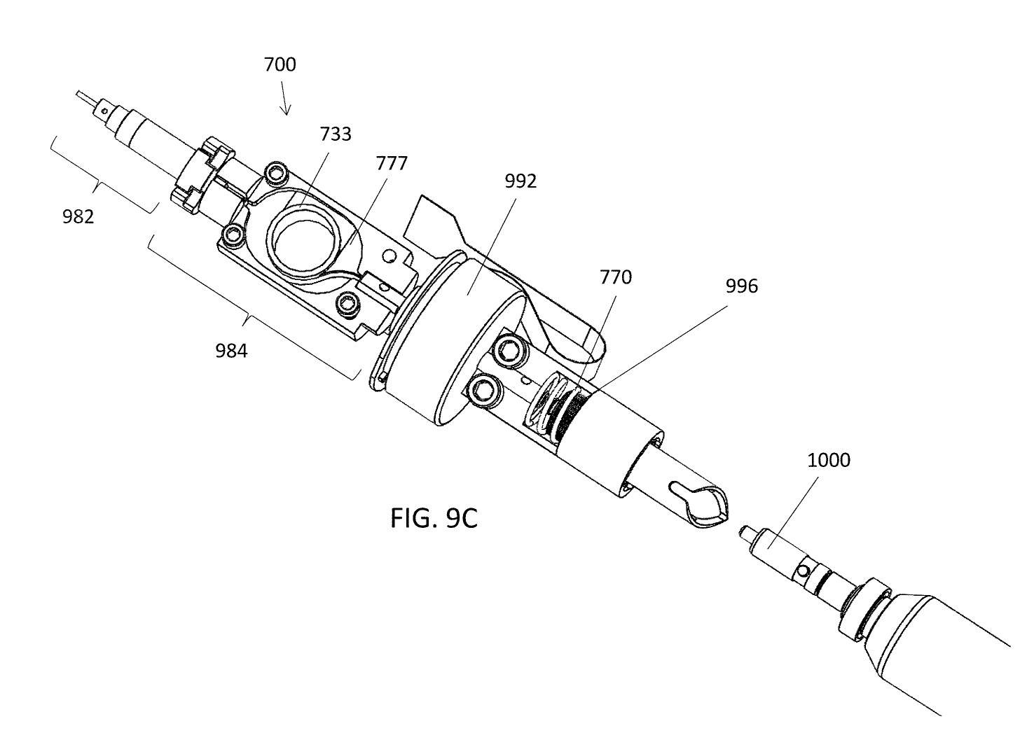

Referring to FIGS. 9A-9C, in some embodiments, the drive assembly 700 can further a rotating optical drive subassembly including a FORJ having a stationary section 982 and a rotatable section 984. A motor 992 can rotate the rotatable section 984. An optical connector 996, such as an FC adaptor, can connect the rotatable section 984 with the rotatable fiber of the catheter 1000 (which can be any catheter described herein). The shaft of the motor 992 can be hollow so as to allow a portion of the rotatable section 984 of the FORJ to extend therethrough (i.e., the motor 992 and the FORJ can be coaxial). When motor 992 rotates, the rotatable section 984 of the FORJ can rotate, thereby causing the optical connector 996 (and thus the driveshaft and optical fiber of the imaging catheter) to rotate when connected. As shown in FIGS. 9A-9C, the fiber management unit 777 can be aligned with the motor 992 and the FORJ and configured to rotate therewith.

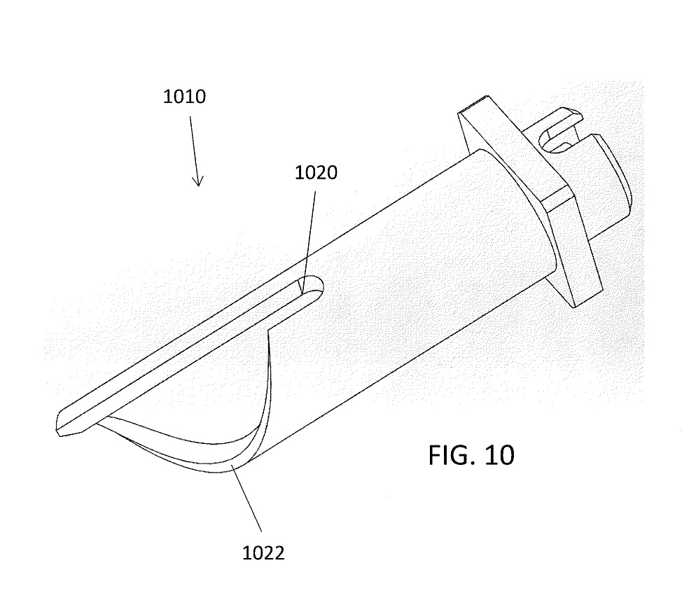

Another exemplary embodiment of a receiving tube 1010 is shown in FIG. 10. The receiving tube 1010 is similar to the other receiving tubes described herein except that it includes only a single curved wall 1022. The wall 1022 curves (e.g., in a helix) from the distal tip of the tube 1010 to the elongated channel 1020. In use, a keyed feature can slide along the single wall 1022 and into the keyed channel 1020.

The alignment and attachment mechanisms systems described herein are configured so that catheter engagement with the drive assembly requires no manual control of the relative orientation between the respective optical connectors by the user. The user is able to load the catheter into the drive assembly, and the mechanisms described herein automatically align the optical fibers as required. Thus, the system described herein allows for engagement of the catheter with the drive assembly where neither optical connectors of either component is required to be in a predetermined configuration (yet the ends, such as the cleaved ends, of the optical fiber can still align properly). The user is not required to manually adjust the connector alignment between the drive assembly and the catheter. In addition, the manufacturing process does not need to include specifically orienting the optical fiber within the catheter and/or drive assembly. Further, when removed from the packaging, no verification of alignment of the connectors is required. In turn, the design of the catheter and/or drive assembly is simplified with the elimination of parts and features intended to orient the optical connector in a specific position.

Although described herein as being used with a catheter, it should be understood that the alignment mechanisms herein can also be used as part of an imaging device that includes, for example, a rod rather than a catheter.

The alignment systems can be used herein to align optical fibers for optical coherence tomography (OCT) and/or to align optical connections for other types of imaging, such as ultrasound.

Further, although described herein as being used with an optical fiber for optical alignment, the alignment mechanisms described herein can alternatively or additionally be used for alignment of other features, such as electrical connections.

Although the alignment mechanisms are described herein as having a catheter with a keyed feature and a drive assembly with a mating channel, in some embodiments, the drive assembly can included the keyed feature, and the catheter can have the mating channel.

In some embodiments, there is tolerance built in to the keyed feature and the mating channel. In such embodiments, another feature, such as a spring, can help ensure proper alignment.

In some embodiments, the optical fiber of the catheter and the optical fiber of the drive assembly are each cleaved at between 5 and 10 degrees, such as 8 degrees. Proper alignment ensures that these cleaved ends are flush with one another.

The alignment and attachment mechanisms described herein can be used with a variety of different driving assemblies. For example, the alignment and attachment mechanisms can be used in addition to or in place of feature in the drive assemblies described in U.S. patent application Ser. No. 14/400,151, filed Nov. 10, 2014, titled “ATHERECTOMY CATHETER DRIVE ASSEMBLIES,” now U.S. Pat. No. 9,345,398, the entirety of which is incorporated by reference herein.

Moreover, the alignment and attachment mechanisms described herein can be used with a variety of different catheters. Exemplary catheters are described in U.S. Patent Application No. 61/646,843, titled “ATHERECTOMY CATHETERS WITH IMAGING,” filed on May 14, 2012, U.S. patent application Ser. No. 13/433,049, titled “OCCLUSION-CROSSING DEVICES, IMAGING, AND ATHERECTOMY DEVICES,” filed Mar. 28, 2012, U.S. patent application Ser. No. 13/175,232, titled “ATHERECTOMY CATHETERS WITH LONGITUDINALLY DISPLACEABLE DRIVE SHAFTS,” filed on Jul. 1, 2011, U.S. patent application Ser. No. 12/829,277, titled “ATHERECTOMY CATHETER WITH LATERALLY-DISPLACEABLE TIP,” filed on Jul. 1, 2010, and U.S. patent application Ser. No. 12/829,267, titled “CATHETER-BASED OFF-AXIS OPTICAL COHERENCE TOMOGRAPHY IMAGING SYSTEM,” filed on Jul. 1, 2010, International Patent Application No. PCT/US2015/014613, titled “ATHERECTOMY CATHETERS AND OCCLUSION CROSSING DEVICES,” filed on Feb. 5, 2015, U.S. patent application Ser. No. 15/072,272, titled “ATHERECTOMY CATHETERS DEVICES HAVING MULTI-CHANNEL BUSHINGS,” filed on Mar. 16, 2016, and U.S. patent application Ser. No. 15/076,568, titled “ATHERECTOMY CATHETERS AND OCCLUSION CROSSING DEVICES,” filed on Mar. 21, 2016, all of which are herein incorporated by reference in their entirety.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent to” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements, these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein is intended to include all sub-ranges subsumed therein.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A self-aligning system for coupling a catheter to a rotational drive assembly, the apparatus comprising:
a catheter comprising:
an elongate catheter body;
a rotatable driveshaft extending within the elongate catheter body;
a connector at a proximal end of the elongate catheter body; and
a keyed feature extending from an outer diameter of the connector; and
a drive assembly comprising:
a motor configured to rotate the driveshaft;
a receiving tube with a distal opening configured to receive the connector;
and
a channel on an inner surface of the receiving tube, the channel including two opposing curved walls that are angled towards a substantially straight section;
wherein the keyed feature is configured to slide against one of the opposing curved walls and into the substantially straight section to rotationally align the proximal end of the elongate catheter body with the drive assembly.

2. A catheter comprising:
an elongate catheter body;
a rotatable driveshaft extending within the elongate catheter body;
a connector at a proximal end of the elongate catheter body; and
a keyed feature extending from an outer diameter of the connector;
wherein the catheter is configured to connect to a drive assembly for rotating the rotatable drive shaft, the keyed feature configured to slide against opposing curved walls of the drive assembly and into a substantially straight section to rotationally align the proximal end of the elongate catheter body with the drive assembly.

3. A drive assembly comprising:
a motor configured to rotate a catheter driveshaft;
a receiving tube with a distal opening configured to receive a connector of a catheter; and
a channel on an inner surface of the receiving tube, the channel including two opposing curved walls that are angled towards a substantially straight section;
wherein the opposing curved walls are configured to receive a keyed feature of the catheter and guide the keyed feature into the substantially straight section to rotationally align the proximal end of the catheter with the drive assembly.

4. The self-aligning system of claim 1, further comprising:
a first optical fiber extending through the elongate catheter body; and
a second optical fiber within the driveshaft configured to transfer light from a light source to the optical fiber of the catheter when optically connected thereto.

5. The self-aligning system of claim 4, wherein rotationally aligning the proximal end of the catheter with the drive assembly optically couples the first optical fiber with the second optical fiber.

6. The self-aligning system of claim 1 or 3, wherein the drive assembly further includes a fiber optic rotating junction having a stationary portion with a stationary fiber therein and a rotatable portion with a rotatable fiber therein.

7. The self-aligning system of claim 6, wherein the motor is configured to rotate the rotatable portion of the fiber optic rotating junction.

8. The self-aligning system of claim 6, wherein the motor is hollow and configured to house a portion of the fiber optic rotating junction of the drive assembly such that the motor and the fiber optic rotating junction are coaxial.

9. The self-aligning system of claim 1 or 3, wherein a distal end of the receiving tube is beveled.

10. The self-aligning system of claim 9, wherein the bevel is at an angle of between 35 and 45 degrees relative to a central longitudinal axis of the receiving tube.

11. The self-aligning system of claim 9, wherein the bevel includes a tip.

12. The self-aligning system of claim 11, wherein the straight section of the channel is approximately 180 degrees from the tip.

13. The self-aligning system of claim 1 or 2, wherein the keyed feature is a cylindrical post extending from the connector.

14. The self-aligning system of claim 13, wherein a central long axis of the cylindrical post is substantially perpendicular to a central long axis of the connector.

15. The self-aligning system of claim 1 or 2, wherein the connector is substantially cylindrical.

16. The self-aligning system of claim 1 or 3, wherein an inner diameter of the receiving tube is larger than an outer diameter of the connector without the keyed feature and smaller than an outer diameter of the connector with the keyed feature.

17. The self-aligning system of claim 1 or 3, wherein an inner surface of the receiving tube further includes a first notched tab and a second notched tab positioned on opposite sides of the substantially straight section.

18. A self-aligning coupling apparatus for coupling an imaging device to a rotational drive assembly, the apparatus comprising:
an imaging device comprising:
an elongate body;
a first optical fiber extending through the catheter body;
a connector at a proximal end of the elongate body; and
a keyed feature extending from an outer diameter of the connector; and
a drive assembly comprising:
a motor configured to rotate the optical fiber;
a second optical fiber configured to transfer light from a light source to the optical fiber of the imaging device when optically connected thereto;
a receiving tube with a distal opening configured to receive the connector;
and
a channel on an inner surface of the receiving tube, the channel including two opposing curved walls that are angled towards a substantially straight section;
wherein the keyed feature is configured to slide against one of the opposing curved walls and into the substantially straight section to align the proximal end of the imaging device with the drive assembly to optically couple the first optical fiber with the second optical fiber.

19. An imaging device comprising:
an elongate body;
an optical fiber extending within the elongate body;
a connector at a proximal end of the elongate body; and
a keyed feature extending from an outer diameter of the connector;
wherein the imaging device is configured to connect to a drive assembly for rotating the optical fiber, the keyed feature configured to slide against opposing curved walls of the drive assembly and into a substantially straight section to rotationally align the proximal end of the elongate body with the drive assembly to optically couple the optical fiber with a second optical fiber of the drive assembly.

20. A drive assembly comprising:
a motor configured to rotate an imaging device;
a receiving tube with a distal opening configured to receive a connector of an imaging device;
an optical fiber configured to transfer light from a light source to an optical fiber of the imaging device when optically connected thereto;
and
a channel on an inner surface of the receiving tube, the channel including two opposing curved walls that are angled towards a substantially straight section;
wherein the opposing curved walls are configured to receive a keyed feature of the imaging device and guide the keyed feature into the substantially straight section to rotationally align the proximal end of the catheter with the drive assembly to optically couple the optical fiber of the drive assembly with the optical fiber of the imaging device.

21. A self-aligning system for coupling a catheter to a rotational drive assembly, the apparatus comprising:
a catheter comprising:
an elongate catheter body;
a rotatable driveshaft extending within the elongate catheter body;
a connector at a proximal end of the elongate catheter body; and
a keyed feature extending from an outer diameter of the connector; and
a drive assembly comprising:
a motor configured to rotate the driveshaft;
a receiving tube with a distal opening configured to receive the connector;
and
a channel on an inner surface of the receiving tube, the channel including a curved wall that spirals to a substantially straight section;
wherein the keyed feature is configured to slide along the curved wall and into the substantially straight section to rotationally align the proximal end of the elongate catheter body with the drive assembly.

22. A catheter comprising:
an elongate catheter body;
a rotatable driveshaft extending within the elongate catheter body;
a connector at a proximal end of the elongate catheter body; and
a keyed feature extending from an outer diameter of the connector;
wherein the catheter is configured to connect to a drive assembly for rotating the rotatable drive shaft, the keyed feature configured to slide against a curved wall of the drive assembly and into a substantially straight section to rotationally align the proximal end of the elongate catheter body with the drive assembly.

23. A drive assembly comprising:
a motor configured to rotate a catheter driveshaft;
a receiving tube with a distal opening configured to receive a connector of a catheter; and
a channel on an inner surface of the receiving tube, the channel including a curved wall that is angled towards a substantially straight section;
wherein the curved wall is configured to receive a keyed feature of the catheter and guide the keyed feature into the substantially straight section to rotationally align the proximal end of the catheter with the drive assembly.

24. A self-aligning coupling apparatus for coupling an imaging device to a rotational drive assembly, the apparatus comprising:
an imaging device comprising:
an elongate body;
a first optical fiber extending through the catheter body;
a connector at a proximal end of the elongate body; and
a keyed feature extending from an outer diameter of the connector; and
a drive assembly comprising:
a motor configured to rotate the optical fiber;
a second optical fiber configured to transfer light from a light source to the optical fiber of the imaging device when optically connected thereto;
a receiving tube with a distal opening configured to receive the connector;
and
a channel on an inner surface of the receiving tube, the channel including a curved wall that is angled towards a substantially straight section;
wherein the keyed feature is configured to slide against the curved wall and into the substantially straight section to align the proximal end of the imaging device with the drive assembly to optically couple the first optical fiber with the second optical fiber.

25. An imaging device comprising:
an elongate body;
an optical fiber extending within the elongate body;
a connector at a proximal end of the elongate catheter body; and
a keyed feature extending from an outer diameter of the connector;
wherein the imaging device is configured to connect to a drive assembly for rotating the optical fiber, the keyed feature configured to slide against a curved wall of the drive assembly and into a substantially straight section to rotationally align the proximal end of the elongate body with the drive assembly to optically couple the optical fiber with a second optical fiber of the drive assembly.

26. A drive assembly comprising:
a motor configured to rotate an imaging device;
a receiving tube with a distal opening configured to receive a connector of an imaging device;
an optical fiber configured to transfer light from a light source to an optical fiber of the imaging device when optically connected thereto;
and
a channel on an inner surface of the receiving tube, the channel including a curved wall that is angled towards a substantially straight section;
wherein the curved wall is configured to receive a keyed feature of the imaging device and guide the keyed feature into the substantially straight section to rotationally align the proximal end of the catheter with the drive assembly to optically couple the optical fiber of the drive assembly with the optical fiber of the imaging device.
